(19) United States
(12) Patent Application Publication
(10) Pub. No.: US 2017/0361050 A1
(43) Pub. Date: Dec. 21, 2017

(54) ADJUSTABLE AIRFLOW DIFFUSER

(71) Applicant: KONINKLIJKE PHILIPS N.V., EINDHOVEN (NL)

(72) Inventor: Patrick FINNEGAN, ELIZABETH, PA (US)

(73) Assignee: Koninklijke Philips N.V., Eindhoven (NL)

(21) Appl. No.: 15/535,420

(22) PCT Filed: Dec. 10, 2015
(86) PCT No.: PCT/IB2015/059506

§ 371 Date: Jun. 13, 2017

Related U.S. Application Data
(60) Provisional application No. 62/093,869, filed on Dec. 18, 2014.

(51) Int. Cl. A61M 16/08; A61M 16/06

(52) U.S. Cl. CPC A61M 16/08; A61M 16/0666

(57) ABSTRACT

An adjustable airflow diffuser for selectively diffusing a flow of treatment gas includes a first member adapted to be disposed in the flow and a second member movably coupled to the first member and also adapted to be disposed in the flow. The second member is selectively moveable from: (i) a first position with respect to the first member in which the first member and the second member form a first turbulating arrangement, and (ii) a second position with respect to the first member in which the first member and the second member form a second turbulating arrangement.

### CROSS-REFERENCE TO RELATED APPLICATIONS

This patent application claims the priority benefit under 35 U.S.C. §119(e) of U.S. Provisional Application No. 62/093,869, filed on Dec. 18, 2014, the contents of which are herein incorporated by reference.

### BACKGROUND OF THE INVENTION

### 1. Field of the Invention

The present invention relates generally to systems for delivering a flow of a treatment gas to the airway of a patient and, more particularly, to adjustable airflow diffusers for use in such systems.

### 2. Description of the Related Art

There are numerous situations where it is necessary or desirable to deliver a flow of breathable gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder such as sleep apnea syndrome, in particular, obstructive sleep apnea (OSA), or congestive heart failure.

Non-invasive ventilation and pressure support therapies involve the placement of a respiratory patient interface device including a patient interface that is typically secured on the face of a patient by a headgear assembly. The patient interface may be, without limitation, a nasal mask that covers the patient's nose, a nasal cushion having nasal prongs that are received within the patient's nares, a nasal/oral mask that covers the nose and mouth, or full face mask that covers the patient's face. It is known to maintain such devices on the face of a wearer by a headgear having one or more straps adapted to fit over/around the patient's head. Because such respiratory patient interface devices are typically worn for an extended period of time, it is important for the headgear to maintain the patient interface in a desired position while doing so in a manner that is comfortable to the patient.

Certain patient interface devices, particularly nasal cushions and pillows-type devices that are engaged directly with the nose of a patient, can provide a stream of breathing gas into each nostril. The streams of breathing gas together form the flow of breathing gas. However, such streams can cause in the patient a sensation of jetting that is uncomfortable. Such jetting can result from the impingement of a narrow jet of breathing gas at relatively high velocity upon a limited region of the patient, and which can result in drying and irritation of such region. Moreover, such impingement can give the patient a sensation that the pressure of the breathing gas is higher than is actually the case, which is likewise undesirable. It thus would be desirable to provide an improved system that overcomes these and other shortcomings.

### SUMMARY OF THE INVENTION

In certain embodiments, the general nature of the invention can be stated as an adjustable airflow diffuser for selectively diffusing a flow of treatment gas. The diffuser comprises: a first member adapted to be disposed in the flow; and a second member movably coupled to the first member and also adapted to be disposed in the flow. The second member is selectively moveable from: (i) a first position with respect to the first member in which the first member and the second member form a first turbulating arrangement, and (ii) a second position with respect to the first member in which the first member and the second member form a second turbulating arrangement.

The second member may be rotatably coupled to the first member.

The first member may include an aperture defined therethrough.

The aperture may have a cross-sectional profile which generally flares from a first width to a second width.

The first member may include a plurality of apertures defined therethrough.

The second member may include an aperture defined therethrough.

The second member may further include a number of textural elements disposed about an outer periphery thereof.

In other embodiments, the general nature of the invention can be stated as a delivery conduit for communicating a flow of breathing gas from a pressure generating device to an interface device. The delivery conduit comprises: a first end adapted to receive the flow of breathing gas from the pressure generating device; a second end adapted to communicate the flow to the interface device; a first member positioned to be disposed in the flow between the first end and the second end; and a second member movably coupled to the first member. The second member is positioned to be disposed in the flow between the first end and the second end. The second member is selectively moveable from: (i) a first position with respect to the first member in which the first member and the second member form a first turbulating arrangement, and (ii) a second position with respect to the first member in which the first member and the second member form a second turbulating arrangement.

The second member may be rotatably coupled to the first member. The first member may include an aperture defined therethrough. The aperture may have a cross-sectional profile which generally flares from a first width to a second width. The first member may include a plurality of apertures defined therethrough. The second member may include an aperture defined therethrough. The second member may further include a number of textural elements disposed about an outer periphery thereof

In further embodiments, the general nature of the invention can be stated as a method of diffusing a flow of breathing gas. The method comprises: providing a first member in the flow of breathing gas; providing a second member in the flow of breathing gas; and enhancing diffusion of the flow of breathing gas by adjusting the relative position of the first member and the second member from: (i) a first position in which the first member and the second member form a first turbulating arrangement, to (ii) a second position in which the first member and the second member form a second turbulating arrangement.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the invention;

FIG. 2 is a side elevation view of an airflow diffuser according to one exemplary embodiment of the invention coupled between a delivery conduit and a coupler for a patient interface device;

FIG. 3A is an elevation view of the downstream side of a first portion of the airflow diffuser of FIG. 2;

FIG. 3B is a sectional view of the first portion of the airflow diffuser shown in FIG. 3A taken along line 3B-3B thereof;

FIG. 4A is an elevation view of the downstream side of second portion of the airflow diffuser of FIG. 2;

FIG. 4B is a sectional view of the second portion of the airflow diffuser shown in FIG. 4A taken along line 4B-4B thereof; and

FIGS. 5A-5C are elevation views of the airflow diffuser of FIG. 2 taken along line 5-5 thereof showing two members thereof disposed in different relative positions with respect to each other.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components.

As used herein, the word “number” means one, or any integer greater than one (i.e., a plurality). As used herein, the terms “turbulating arrangement” means any arrangement which increases turbulence in a flow of breathing gas.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein. Similar numerals refer to similar parts throughout the specification.

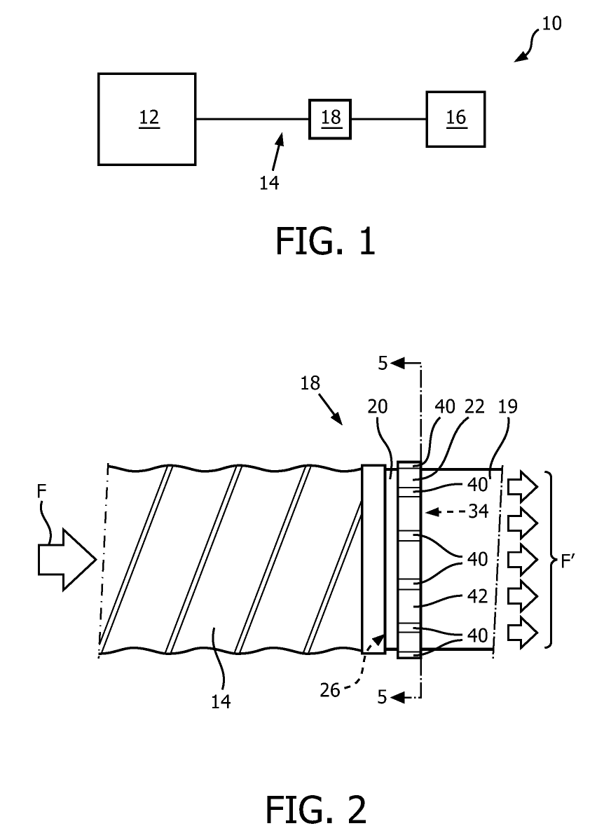

A system 10 adapted to provide a regimen of respiratory therapy to a patient according to one exemplary embodiment of the invention is shown schematically in FIG. 1. System 10 includes a pressure generating device 12, a delivery conduit 14, and a patient interface device 16. System 10 further includes an adjustable airflow diffuser 18 fluidly coupled between pressure generating device 12 and patient interface device 16 as a portion of delivery conduit 14. While in the exemplary embodiment illustrated in FIG. 1, adjustable airflow diffuser 18 is shown as an independent element provided along delivery conduit 14, it will be understood that that is but one possible, exemplary implementation of the present invention. It will thus be appreciated that other, alternative implementations are also possible, such as, without limitation, direct coupling of airflow diffuser 18 to an elbow conduit or other suitable member (not shown) which is coupled to patient interface device 16 or direct coupling of airflow diffuser 18 to patient interface device 16.

Pressure generating device 12 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 14 is structured to communicate the flow of breathing gas from pressure generating device 12 to patient interface device 16 and as such may be formed from any suitable construction for carrying out such purpose.

Patient interface device 16 may be, without limitation, a nasal mask that covers the patient's nose, a nasal cushion having nasal prongs that are received within the patient's nares, a nasal/oral mask that covers the patient's nose and mouth, or a full face mask that covers the patient's face. Patient interface device 16 may be secured to the patient via any suitable means without varying from the scope of the present invention.

FIG. 2 shows a side elevation view of an airflow diffuser 18 according to one exemplary embodiment of the invention coupled between a delivery conduit 14 and a coupler 19 (e.g., without limitation, an elbow) for a patient interface device. Airflow diffuser 18 includes a first member 20 and a second member 22, each formed from a medical grade plastic or other suitable material, disposed in a flowpath of a flow F of breathing gas passing through delivery conduit 14. Second member 22 is movably coupled to first member 20 either directly, such as illustrated, or through one or more intermediate elements, the purpose of which will be discussed in greater detail below.

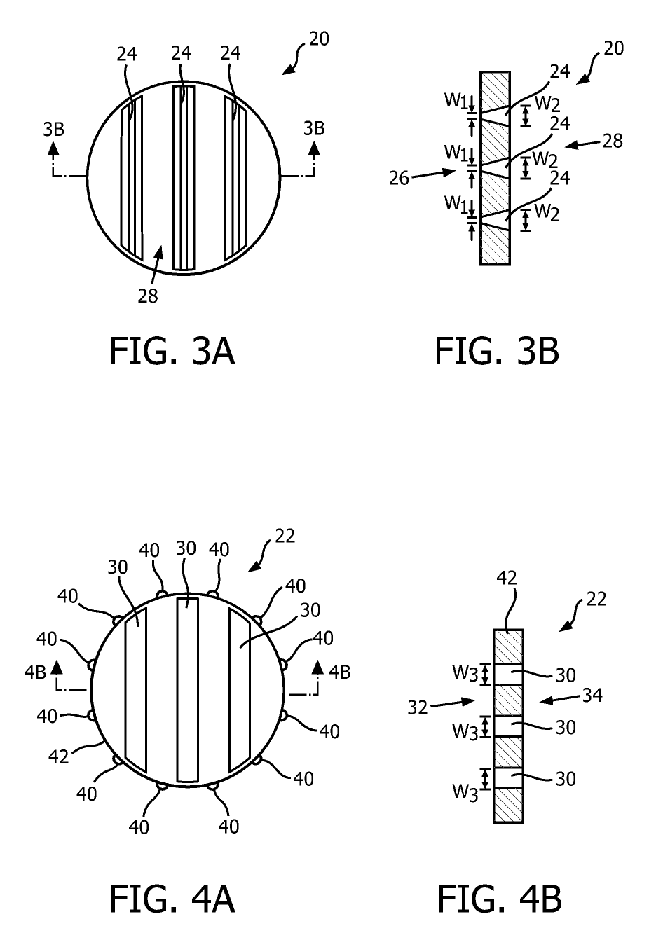

Referring to FIGS. 3A and 3B, first member 20 is generally disc-shaped and includes a number of apertures 24 defined therethrough. In the illustrated embodiment, first member 20 includes three apertures 24, with each aperture having a cross-sectional profile which generally flares from a first width w₁ on an upstream side 26 to a second width w₂ on a downstream side 28. It is to be appreciated, however, that one or more of the size, shape, placement, cross-sectional profile, or quantity of apertures may be varied without varying from the scope of the present invention. It is also to be appreciated that the shape of first member 20, as well the inclusion of any aperture formed therethrough, may be varied without varying from the scope of the invention (i.e., first aperture 20 may be void of any aperture(s) in particular embodiments).

Referring to FIGS. 4A and 4B second member 22 is also generally disc-shaped and includes a number of apertures 30 defined therethrough. In the illustrated embodiment, second member 22 includes three apertures 30, with each aperture having a constant cross-sectional profile having a constant width w₃ extending from an upstream side 32 to a downstream side 34. Second member 22 may further include a number of textural elements 40 disposed about the outer periphery 42 thereof formed integrally from the same material as second member 22 or alternately from another suitable material coupled to second member 22. The purpose of textural elements 40 is discussed in detail below. Although shown as having an equal number of apertures as first member 20, it is to be appreciated, however, that the quantity of apertures defined through second member 22 may be varied independent of first member 20 without varying from the scope of the present invention. Additionally, it is to be appreciated that one or more of the size, shape, placement or cross-sectional profile of apertures formed in second member 22 may be varied without varying from the scope of the present invention. It is also to be appreciated that the shape of second member 22, as well the inclusion of any aperture formed therethrough, may be varied without varying from the scope of the invention (i.e., second member 22 may be void of any aperture(s) in particular embodiments).

Having thus described the basic structure of each of the first and second members 20 and 22, the basic operation thereof in airflow diffuser 18 will now be discussed. As shown in FIG. 2, airflow diffuser 18 is positioned such that flow F of treatment gas first encounters upstream side 26 of first member 20. Flow F then passes through apertures 24 of first member 20, which introduces turbulence to flow F, and thus diffuses flow F into diffused flow F′ (such as shown in FIG. 2) which then proceeds onward to the patient interface device and the airway of the patient. The turbulence introduced into flow F can be adjusted by selectively adjusting the relative positioning of first member 20 and second member 22 with respect to each other. As previously discussed, second member 22 is movably coupled to first member 20 and, more particularly, in the illustrated example is rotatably coupled to first member such that the relative (angular) positioning of first member 20 and second member 22 may be selectively adjusted by rotating second member 22. Referring to FIG. 2, second member 22 may be rotated by simply grasping outer periphery 42 thereof, and if needed for extra grip, textural elements 40 thereof

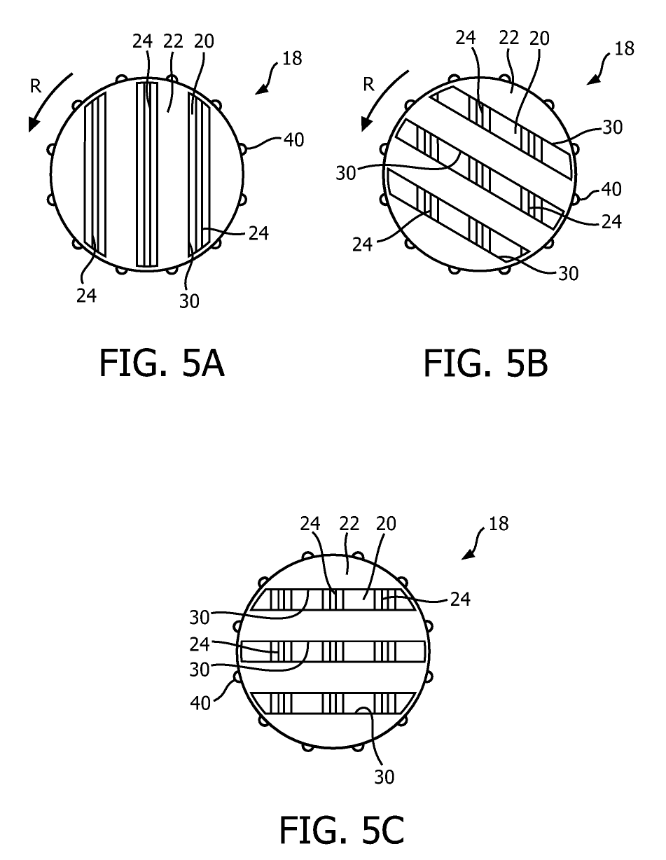

FIGS. 5A-5C show three different arrangements of airflow diffuser 18 in which first member 20 and second member 22 are disposed in different relative positions with respect to each other. For example, FIG. 5A shows an arrangement in which first member 20 and second member 22 are generally positioned relatively such that the apertures 24, 30 of each are generally aligned. FIG. 5B shows an arrangement in which first member 20 and second member 22 are generally positioned relatively such that the apertures 24, 30 of each are partially misaligned, such as if second member 22 were rotated counterclockwise (such as shown by arrow R) with respect to first member 20 from the position shown in FIG. 5A. FIG. 5C shows another arrangement in which first member 20 and second member 22 are generally positioned relatively such that the apertures 24, 30 of each are further misaligned, such as if second member 22 were rotated further counterclockwise (such as shown by arrow R) with respect to first member 20 from the position shown in FIG. 5B.

It thus can be seen that embodiments of the invention provide a user (e.g., a patient) with the ability to selectively enhance (or decrease) the turbulence of the flow of breathing gas, which is desirable from a therapeutic standpoint. That is, such enhanced turbulence increases comfort to the patient by reducing localized dryness and a reduced sense of high pressure to the airway or adjacent portions of the user. Thus, embodiments of the invention provide a user with a readily customizable aspect to an otherwise typically strict therapy experience. Other benefits will be apparent to one of ordinary skill in the art.

Although shown being rotatable with respect to each other, it is to be appreciated that embodiments of the invention also may include elements which are moveable in other directions such that the relative positioning of such elements with respect to each other may be varied. For example, without limitation, elements which are moveable to change one or more of a relative separation distance, offset, or other characteristic of the arrangement may be employed without varying from the scope of the present invention.

While the present invention has been described in connection with a patient interface device used to treat, for example, a patient suffering from OSA, it will be understood that that is meant to be exemplary, and that the principles of the present invention can be can also be applied in connection with other non-treatment applications where a mask receiving a pressurized flow of a breathing gas is employed.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. An adjustable airflow diffuser for selectively diffusing a flow (F) of treatment gas being provided to a patient interface device from a pressure generating device, the diffuser comprising:
a first member adapted to be disposed in the flow; and
a second member movably coupled to the first member, the second member adapted to be disposed in the flow, wherein the second member is selectively moveable from (i) a first position with respect to the first member in which the first member and the second member form a first turbulating arrangement, and (ii) a second position with respect to the first member in which the first member and the second member form a second turbulating arrangement.

2. The adjustable airflow diffuser of claim 1, wherein the second member is rotatably coupled to the first member.

3. The adjustable airflow diffuser of claim 1, wherein the first member includes an aperture defined therethrough.

4. The adjustable airflow diffuser of claim 3, wherein the aperture has a cross-sectional profile which generally flares from a first width (w₁) to a second width (w₂).

5. The adjustable airflow diffuser of claim 1, wherein the first member includes a plurality of apertures defined therethrough.

6. The adjustable airflow diffuser of claim 3, wherein the second member includes an aperture defined therethrough.

7. The adjustable airflow diffuser of claim 1, wherein the second member further includes a number of textural elements disposed about an outer periphery thereof.

8. A delivery conduit for communicating a flow (F) of breathing gas from a pressure generating device to an interface device, the delivery conduit comprising:
a first end adapted to receive the flow of breathing gas from the pressure generating device;
a second end adapted to communicate the flow to the interface device;
a first member positioned to be disposed in the flow between the first end and the second end; and
a second member movably coupled to the first member, the second member positioned to be disposed in the flow between the first end and the second end, the second member being selectively moveable from (i) a first position with respect to the first member in which the first member and the second member form a first turbulating arrangement, and (ii) a second position with respect to the first member in which the first member and the second member form a second turbulating arrangement.

9. The delivery conduit of claim 8, wherein the second member is rotatably coupled to the first member.

10. The delivery conduit of claim 8, wherein the first member includes an aperture defined therethrough.

11. The delivery conduit of claim 10, wherein the aperture has a cross-sectional profile which generally flares from a first width (w₁) to a second width (w₂).

12. The delivery conduit of claim 8, wherein the first member includes a plurality of apertures defined therethrough.

13. The delivery conduit of claim 10, wherein the second member includes an aperture defined therethrough.

14. The delivery conduit of claim 8 wherein the second member further includes a number of textural elements disposed about an outer periphery thereof.

15. A method of diffusing a flow of breathing gas being provided to a patient interface device from a pressure generating device, the method comprising:
providing a first member in the flow of breathing gas;
providing a second member in the flow of breathing gas; and
enhancing diffusion of the flow of breathing gas by adjusting the relative position of the first member and the second member from: (i) a first position in which the first member and the second member form a first turbulating arrangement, to (ii) a second position in which the first member and the second member form a second turbulating arrangement.
